(19) United States
(12) Patent Application Publication
(10) Pub. No.: US 2025/0001108 A1
(43) Pub. Date: Jan. 2, 2025

(54) Displaying A Maintenance Reminder On An Apparatus For Supplying Medical Nitric Oxide

(71) Applicant: INOSYSTEMS, Antony (FR)

(72) Inventors: Yann BLANDIN, Antony (FR); Mary SCHMITT, Antony (FR); Frederic MARCHAL, Antony (FR); Nathan PROUVEZ, Antony (FR)

(73) Assignee: INOSYSTEMS, Antony (FR)

(21) Appl. No.: 18/752,962

(22) Filed: Jun. 25, 2024

(30) Foreign Application Priority Data
Jun. 29, 2023 (FR) 2306904

(51) Int. Cl. A61M 16/00; A61M 16/12

(52) U.S. Cl. CPC A61M 16/0051; A61M 16/12; A61M 2202/0275; A61M 2205/3317; A61M 2205/502; A61M 2205/582; A61M 2205/70

(57) ABSTRACT

The invention relates to an apparatus (1) for supplying an NO-containing gas, comprising an internal circuit (6) for conveying a flow of NO-containing gas, comprising valve means (7), control means (8) with a microprocessor (9), a tactile graphical display screen (4), and a gas analysis line (111) comprising at least one sensor (112). The control means (8) are configured to control, at a given time frequency, a temporary display, on the graphical display screen (4), of a first virtual selection key (81), the actuation of which by a user initiates a procedure of calibration of the sensor or sensors (112) of the gas analysis line (111).

### TECHNICAL FIELD

The invention relates to an apparatus or device for supplying and monitoring a gas containing nitric oxide (NO), in particular an NO/N₂ gas mixture, commonly referred to as an NO supply apparatus, used to treat one or more persons suffering from acute pulmonary arterial hypertension, and configured to display a maintenance reminder, preferably on a daily basis.

### BACKGROUND

Inhaled nitric oxide (NOi) is a standard of care for treating persons, i.e. patients, suffering from acute pulmonary arterial hypertension. Indeed, when inhaled, NO dilates the pulmonary vessels and increases oxygenation by improving gas exchange. These properties are used to treat various medical conditions such as persistent pulmonary hypertension of the newborn (PPHN), acute respiratory distress syndrome (ARDS) observed mainly in adults, or pulmonary hypertension (PH) in heart surgery, observed in adults or children, as described in particular by EP-A-560928, EP-A-1516639 and U.S. Pat. No. 10,201,564.

Usually, a small quantity of gaseous NO (i.e. a few ppm vol.), diluted in nitrogen (N₂), is injected and diluted in a gas flow containing oxygen, typically at least 20 to 21% vol. of oxygen (O₂), such as an N₂/O₂ mixture or air, or even pure oxygen, which is conveyed through the patient circuit of a gas supply installation, and the final gas mixture obtained, containing NO and oxygen, is then inhaled by the patient. The final concentration of NO, which corresponds to a dosage, is determined by the doctor or the like. In general, it is between 1 and 80 ppm by volume (ppmv), typically of the order of 10 to 20 ppmv, depending on the population treated, i.e. neonates, children, adolescents or adults, and on the disease to be treated, in the final NO/N₂/O₂ gas administered by inhalation to the patient in question, that is to say after injection of the NO/N₂ mixture into the oxygen-containing gas flow (i.e. approximately >21% vol.) conveyed by the patient circuit.

Implementation of NOi treatment usually involves one or more NO/N₂ cylinders (or an NO generator), an apparatus for supplying and monitoring NO, a medical ventilator, and a patient kit comprising a patient circuit and respiratory interface, such as a tracheal intubation tube, or other elements, such as a gas humidifier or the like. Such installations are described in particular by US2022/106189, EP2581103 and EP0839546.

All of these pieces of equipment have to be ready for urgently starting the NOi treatment as soon as it has been prescribed by the doctor. Knowing that the time for treating a patient can be from a few hours to several days, it is essential to regularly check their proper functioning in order to guarantee the smooth progress of the treatment, and this under conditions of optimal safety for the patient. Any unexpected interruption of treatment exposes the patient to serious risks that could jeopardize their survival.

Thus, verifying the proper functioning of the NO supply apparatus is particularly important, since it makes it possible to monitor and control/adjust the amount of NO supplied to the patient during the treatment with NOi. In particular, it is essential to regularly calibrate the monitoring or analysis cells, that is to say the NO, NO₂ and O₂ sensors used for the NO and NO₂ content measurements, and the inspired oxygen fraction FiO₂ in the gas administered to the patient in order to ensure accurate measurements by these cells, typically of the dose of NO administered to the patient and the absence (or near absence) of toxic NO₂ and the proportion of oxygen. Maintaining the accuracy of these measurements is a major safety aspect.

However, the healthcare teams (i.e. doctors, nurses or other healthcare personnel) working in the resuscitation and surgical units of hospitals, where NOi is administered, run through the care and medical procedures on a daily basis, which can lead to the omission of certain monitoring tasks.

A problem is therefore to be able to minimize the risk that healthcare teams, using NOi to treat patients requiring same, fail to perform some or all of the essential checks on the proper functioning of the NO supply apparatus, that is to say the apparatus delivering the NO-containing gas flow, in particular a regular calibration of the monitoring cells, that is to say of the NO, NO₂ and O₂ sensors.

### SUMMARY

A solution of the invention thus relates to an apparatus for supplying (i.e. delivering) an NO-containing gas, also called an NO supply apparatus or NO delivery apparatus, comprising:
an internal gas circuit, e.g. an internal gas circuit for conveying a flow of NO-containing gas, comprising valve means,
microprocessor-based control means, i.e. a controller or similar,
a tactile graphical display screen controlled by the control means, and
a gas analysis line and measuring cells comprising at least one sensor electrically connected to the control means.

Moreover, the control means are configured to control, at a given time frequency, a temporary display, on the graphical display screen, of at least a first virtual selection key, the actuation of which by a user causes initiation, by the control means, of a procedure of calibration of the one or more sensors of the gas analysis line.

Depending on the embodiment under consideration, the apparatus of the invention can comprise one or more of the following features:
the internal gas circuit comprises one or more gas passages.
the gas analysis line is also called the “calibration line”, “monitoring line” or “sampling line”.
the apparatus of the invention is configured to monitor and supply NO gas, i.e. NO-containing gas, such as an NO/N₂ mixture.
the sensor calibration procedure begins after the user has actuated the first virtual selection key.
the first virtual selection key cooperates with the control means in order to supply them with at least one control signal corresponding to a choice made by the user corresponding to the initiation of the sensor calibration procedure.
the control means are further configured to control a display, on the graphical display screen, of at least one item of information relating to one or more safety checks to be carried out, in particular the sensor calibration procedure.
it comprises storage means, such as a computer memory, in particular a flash memory or the like.
the safety check procedure or procedures are stored within the storage means, in particular the sensor calibration procedure.
the storage means are configured to store the safety check procedure(s), in particular the sensor calibration procedure.
the safety check procedures include the procedure of calibration of the one or more sensors of the gas analysis line.
the control means are configured to control the display of said at least the first virtual selection key at a time frequency of 1 to 4 times a day, more preferably 1 or 2 times a day.
the control means are configured to control the display of said at least first virtual selection key at a time frequency of at least once a day:
   either at a fixed time, for example at 08:00 a.m. every day,
   or when the apparatus is switched on, i.e. when the apparatus is started up,
   or after a predefined period of time from the start of administration by the device, for example 24 hours after initiation of the treatment.
   or after a predefined period of time after the last calibration performed, for example 24 hours after the last calibration that was performed.
the control means are configured to control, in addition, a display of a second virtual selection key, i.e. a second tactile key, simultaneously or successively to said first virtual selection key.
said second virtual selection key is configured to cancel or reject said sensor calibration procedure, in response to an actuation by the user of the second virtual selection key.
the second virtual selection key cooperates with the control means in order to supply them with at least one control signal corresponding to a choice made by the corresponding user to cancel or reject said calibration procedure, i.e. to defer it.
the control means are configured to control a simultaneous display of said at least one item of information and of the first and optionally second virtual selection keys.
the control means are configured to control a display, within a pop-up window, of said at least one item of information and of the first virtual selection key and optionally of the second virtual selection key.
the control means are further configured to control a display, on the graphical display screen, of a third virtual selection key, the actuation of which by the user causes a discontinuation of a calibration procedure that has started, that is to say that has started after the user has pressed the first virtual selection key.
the control means are further configured to control a display, on the graphical display screen, of a bar graph, an hourglass or an analogous time graph representing the time that has elapsed since the start of the calibration procedure and/or the time remaining before the termination, i.e. end, of the calibration procedure.
the control means are further configured to control a display, on the graphical display screen, of the time graph representing the time that has elapsed since the start of the calibration procedure and/or the time remaining before the termination of the calibration procedure, in particular a time graph representing a bar graph or an hourglass.
the control means are further configured to effect a simultaneous display, on the graphical display screen, of the third virtual selection key and of the time graph, i.e. the display of the third virtual selection key and that of the time graph, i.e. hourglass, bar graph or the like, are effected simultaneously.
it comprises an internal gas analyser comprising the gas analysis line and said at least one sensor, i.e. one or more sensors, preferably an NO sensor and an NO₂ sensor, and preferably an O₂ sensor.
the internal circuit allows the flow of NO-containing gas to be conveyed between (at least) a gas inlet and (at least) a gas outlet supplying the NO-containing gas, i.e. an NO/N₂ mixture.
the internal circuit is part of a gas circuit arranged in the NO supply apparatus.
said at least one sensor comprises one or more electrochemical cells.
it comprises an NO sensor configured to measure a concentration (i.e. content or dose) of NO.
it comprises an NO₂ sensor configured to measure a concentration (i.e. content or dose) of NO₂.
it comprises an O₂ sensor configured to measure a concentration (i.e. content or dose) of O₂.
the one or more sensors are arranged so as to carry out concentration measurements within the gas analysis line.
said internal circuit comprises one or more ducts, passages, tubes or similar.
the gas analysis line comprises ducts, passages, tubes or similar.
the microprocessor-based control means comprise (at least) an electronic board.
the control means comprise one or more microprocessors arranged on the electronic board.
the tactile graphical display screen is part of a graphical user interface (GUI).
the graphical display screen shows a colour display.
the graphical display screen is configured to detect contact of a user's finger pressing on the surface of the graphical display screen.
the graphical display screen comprises a touch panel.
the first and second virtual selection keys are tactile keys displayed on the graphical display screen.
the first and second virtual selection keys cooperate with the control means.
the control means act in response to any pressing of a user's finger on one and/or the other of the first and second selection keys.
they comprise a time counter.
the control means integrate or cooperate with the time counter.
the time counter is integrated in the microprocessor.
the display is effected automatically at the given frequency, typically every 24 hours, that is to say once a day at least, being controlled by the control means which cooperate with the time counter.
it comprises selection means for fixing, setting or selecting a desired amount of NO, i.e. a content or concentration of NO.
the selection means comprise at least one key or one button, in particular a virtual key displayed on the graphical display screen.
the valve means comprise at least one solenoid valve, preferably one or more proportional solenoid valves.
the valve means, in particular the one or more solenoid valves, are controlled by the control means to control the flow of gas within said internal gas circuit.

The invention also relates to an installation for administering gas to a patient, i.e. an NO-containing gas, comprising:
at least one gas source containing gaseous NO, in particular an NO/N₂ gas mixture,
a gas supply apparatus according to the invention, fed with NO-containing gas by said at least one gas source,
a medical ventilator for supplying a gas containing oxygen, and
a feed line supplied with gas by the gas supply apparatus according to the invention (i.e. with NO-containing gas, such as an NO/N₂ mixture) and by the medical ventilator (i.e. with gas containing oxygen, e.g. air or an O₂/N₂ mixture).

According to the embodiment considered, the gas-administering installation of the invention can comprise one or more of the following features:
the medical ventilator, that is to say a ventilation assistance apparatus, is in fluidic communication with the feed line in order to supply said feed line with a respiratory gas containing at least approximately 21% by volume of oxygen, in particular air or an N₂/O₂ mixture.
the medical ventilator is a respiratory assistance apparatus supplying the gas at constant pressure or, alternatively, the medical ventilator is an HFO (high-frequency oscillation) ventilator delivering the gas by high-frequency oscillations.
the gas feed line is supplied with a therapeutic gas, preferably an NO/N₂ mixture, by the gas supply apparatus and with a respiratory gas containing at least approximately 21% by volume of oxygen, preferably air or an N₂/O₂ mixture, by the medical ventilator.
the gas source contains an NO/N₂ gas mixture containing less than 2000 ppm by volume of NO, the remainder being nitrogen, preferably less than 1000 ppm by volume of NO, the remainder preferably being nitrogen, the therapeutic gas source contains an NO/N₂ mixture containing from 250 to 900 ppm by volume of NO, the remainder being nitrogen, for example of the order of 800 ppm by volume of NO, the remainder being nitrogen.
it further comprises a gas humidifier arranged on the gas feed line, preferably downstream of the point at which the device for supplying therapeutic gas is fluidically connected to said gas feed line, so as to feed it with therapeutic gas.
it further comprises a line for recovering the gases exhaled by the patient.
the gas feed line and the exhaled gas recovery line are connected at a connecting piece, preferably a Y-piece, and define or form all or part of a patient circuit.
the feed line forms an inspiratory branch of the patient circuit.
the exhaled gas recovery line forms an expiratory branch of the patient circuit.
the gas analysis line of the NO supply apparatus is fluidically connected to the gas feed line, i.e. the inspiratory branch.
the gas feed line, i.e. the inspiratory branch, comprises a flow sensor arranged between the ventilator and the site of injection of the NO-containing gas coming from the NO supply apparatus.
the flow sensor is connected to the control means of the NO supply apparatus.
the flow sensor measures the flow of oxygen-containing gas delivered by the medical ventilator and circulating in the gas feed line, i.e. the inspiratory branch.
the gas feed line, i.e. the inspiratory branch, is fluidically connected to an outlet port of the medical ventilator so as to collect and convey the gas delivered by the medical ventilator.
the exhaled gas recovery line, i.e. the expiratory branch, is fluidically connected to an inlet port of the medical ventilator so as to convey, to the medical ventilator, all or some of the gases exhaled by the patient.
at least one source of therapeutic gas comprises one or more gas containers, particularly one or more pressurized-gas cylinders.
the one or more gas containers are equipped with a gas distribution valve with or without an integrated pressure reducer (RDI).
the gas distribution valve is made of copper alloy, such as brass, and/or is equipped with a protective cowl arranged around the gas distribution valve, for example made of polymer material (i.e. plastic), metal or combinations thereof.
the one or more fluid containers are pressurized gas cylinders containing, when full, a gas mixture, in particular NO/N₂, at a pressure of at least 150 to 200 bar abs, or at least 250 to 300 bar abs.
the fluid container has a cylindrical, in particular ogival, general shape.

In general, within the scope of the invention:
“ppmv” signifies parts per million by volume,
“% vol.” signifies percentage by volume,
“NO” means nitric oxide.
“NO₂” means nitrogen dioxide.
“N₂” means nitrogen.
“O₂” means oxygen.
“sensor” means a measuring device designed for and suitable and/or configured for measuring and providing one or more signals or measurements; for example, an NO sensor is an NO concentration measurement device for measuring and supplying one or more nitric oxide concentration signals or measurements.
the terms “concentration”, “dose” and “content” shall be considered as equivalents.
the terms “means of/to/for” are considered to be wholly equivalent to and capable of being substituted by the terms “device of/to/for”, for example the terms “control means” may be replaced by “control device”, the terms “valve means” may be replaced by “valve device”, the “control means” may be replaced by “control device”, etc. . . .

### BRIEF DESCRIPTION OF THE VIEWS OF DRAWINGS

The invention will now be better understood from the following detailed description, given as a non-limiting example, with reference to the appended figures, in which:

FIG. 1 shows an embodiment of an installation for administering gas, i.e. an NO-containing gas, comprising an NO supply apparatus according to the invention.

FIG. 2 schematically shows the operation of an NO supply apparatus according to the invention.

FIG. 3 schematically shows an example of the display of information, relating to safety checks to be carried out, and of a first and a second virtual selection key on the graphical display screen of the NO supply apparatus according to the invention.

FIG. 4 schematically shows an example of the display of a third virtual selection key on the graphical display screen of the NO supply apparatus according to the invention.

### DETAILED DESCRIPTION

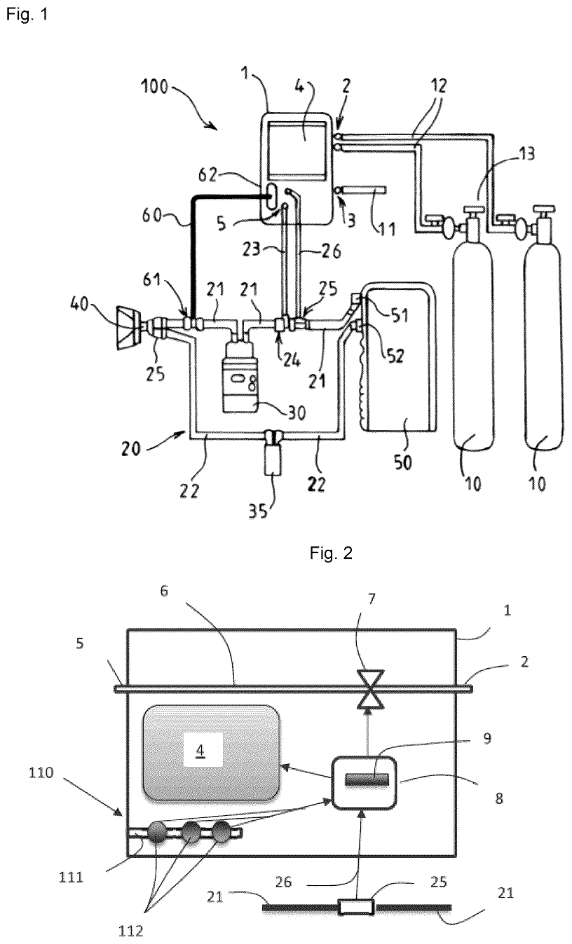

An embodiment of an installation 100 for administering gas is illustrated in FIG. 1. Here, it comprises two pressurized gas cylinders or gas sources 10, each containing an NO/N₂ gas mixture containing up to 1% vol. of NO, typically between 100 and 1500 ppmv of NO (remainder N₂), namely here an NO/N₂ gas mixture containing, for example, 450 or 800 ppmv of NO (remainder N₂), which feed an NO/N₂ mixture to a device or apparatus 1 for supplying, i.e. delivering, NO, making it possible to monitor/follow and control the supply of the NO/N₂ gas mixture.

The gas cylinders 10 are fluidically connected to the NO supply apparatus 1 via gas feed lines 12, such as flexible hoses or conduits or the like, which may be equipped with devices for regulating and/or monitoring the gas pressure, such as gas regulator 13, pressure gauges, etc. The gas feed lines 12 are connected to one or more gas inlets 2 of the NO delivery device 1, which supply an internal gas circuit, for example one or more gas passages internal to the NO delivery apparatus 1, serving to convey the gas within the NO delivery device 1, that is to say in the housing or the outer shell of the NO delivery device 1.

The NO delivery device 1 also comprises an oxygen inlet 3 fluidically connected, via an oxygen feed line 12 such as a flexible hose or the like, to an oxygen source, for example a pressurized oxygen cylinder or a hospital network, that is to say an oxygen supply line arranged in a hospital building.

The installation 100 for administering gas further comprises a medical ventilator 50, that is to say a respiratory assistance apparatus, which supplies a respiratory gas flow containing oxygen, typically of the order of at least about 21% oxygen, such as air or an oxygen/nitrogen (N₂/O₂) mixture, or even oxygen.

The medical ventilator 50 and the NO supply apparatus 1 of the installation 100 for administering gas are in fluidic communication with a gas feed line or inspiratory branch 21 of a patient circuit 20. The gas feed line or inspiratory branch 21 serves to convey the gas flow to the patient, which flow is formed by mixing the oxygen-based flow (i.e. air or NO/N₂ mixture) from the medical ventilator 50 and the NO-containing flow, i.e. the NO/N₂ gas mixture, delivered by the NO supply apparatus 1.

More precisely, the NO supply device 1 delivers or injects the NO/N₂ mixture, for example at 450 or 800 ppmv of NO, into the gas feed line 21 via an injection line or duct 23 connected to an NO outlet 5 of the apparatus 1, in order to mix (at 24.1) the NO/N₂ flow with the oxygen-based gas flow (with at least approximately 21% O₂), e.g. air or an oxygen/nitrogen mixture, delivered by the medical ventilator 50 and conveyed via the inspiratory branch 21 of the patient circuit 20, so as to obtain a final mixture essentially containing NO at the desired dosage, nitrogen (N₂) and oxygen (O₂), and possibly inevitable impurities (e.g. argon, CO₂, NO₂, etc.), that is to say an NO/N₂/O₂ gas mixture.

The inspiratory branch 21 further comprises a gas humidifier 30 arranged downstream of the site 24 where NO is injected into the inspiratory branch 21. It makes it possible to humidify the gas flow, e.g. the NO/N₂/O₂ gas mixture, before it is inhaled by the patient to be treated by way of a respiratory interface 40, such as a tracheal intubation tube, a breathing mask or similar.

There is also provided a line for recovering the gases exhaled by the patient, forming the expiratory branch 22 of the patient circuit 20. The gas feed line or inspiratory branch 21 and the exhaled gas recovery line or expiratory branch 22 are connected at a connection piece 25, preferably a Y-piece.

The inspiratory branch 21 is fluidically connected upstream to an outlet port 51 of the medical ventilator 50, such as a connector, coupling or the like, so as to recover and convey the oxygen-based gas, typically air or N₂/O₂ mixture (containing approximately 21% O₂) delivered by the medical ventilator 50, while the expiratory branch 22 conveying the exhaled gases is fluidically connected to an inlet port 52 of the medical ventilator 50, such as a connector, coupling or the like, so as to return to the medical ventilator 50 all or part of the flow of the gases exhaled by the patient.

The expiratory branch 22 for the exhaled gases can comprise one or more optional components, for example a CO₂ removal device 35, i.e. a CO₂ trap, such as a hot container or the like, used to remove the CO₂ present in the patient's exhaled gases, or a filter or the like.

A flow rate sensor 25, for example of the hot wire or pressure differential type, is arranged on the gas feed line or inspiratory branch 21, between the ventilator 50 and the humidifier 30, and is connected to the NO delivery device 1 via a flow rate measurement line 26. This arrangement serves to measure the flow rate of gas delivered by the ventilator 50, such as air or an N₂/O₂ mixture, and circulating in the inspiratory branch 21, upstream of the site 24 where the injection conduit or line 23 is connected and where the NO/N₂/O₂ gas mixture is formed. This makes it possible to regulate more efficiently the delivery of the NO flow (i.e. N₂/O₂) by the NO delivery device 1, since the flow rate measurements performed by the flow rate sensor 25 are returned, via the flow rate measurement line 26, to the control means of the NO delivery apparatus 1.

As is shown schematically in FIG. 2, the NO supply apparatus 1 comprises, in a conventional manner, a rigid housing, for example made of polymer, through which an internal gas circuit 6 passes, such as a gas conduit or the like, in order to convey the NO/N₂ flow fed through the one or more gas feed lines 33, the latter being supplied by the NO/N₂ mixture cylinders 31. The internal gas circuit 6 fluidically connects the gas inlet (or inlets) 21 of the NO supply apparatus 1 to the injection line 23 via an NO outlet 5 of the apparatus 1, in such a way as to convey the NO-based gas flow between them. The internal gas circuit 6 is arranged in the housing.

Flow control means, typically valve means 7, i.e. one or more valve devices, for example a solenoid valve or a plurality of solenoid valves arranged in parallel, preferably one or more proportional (solenoid) valves, are arranged on the internal gas circuit 6, typically an internal gas passage, in order to control the gas flow(s), i.e. the flow rate, circulating therein in the direction of the injection line 24.

To do this, the valve means 7 are controlled by control means 8, i.e. one or more control devices or controllers, also arranged in the housing of the NO supply apparatus 1, typically an electronic board comprising one or more microprocessors 9, typically one or more microcontrollers, implementing one or more algorithms. They can comprise other elements, such as storage means (not shown), for example a computer memory or computer memories, such as a flash memory. The control means 8 make it possible in particular to adjust or control the gas flow rate by controlling the valve means, typically to open or close said valve or valves, in order to obtain a gas flow rate determined and/or calculated by the control means 8 from a value set/fixed by the user, and as a function of the flow rate of gas, i.e. air, delivered by the ventilator 50 and measured by the flow rate sensor 25 arranged on the inspiratory branch 21 and connected to the NO supply device 1 by the flow rate measurement line 26.

The internal gas circuit 6, in particular an internal gas passage, of the NO supply apparatus 1 can also comprise one or more flow meters (not shown) and/or a pressure regulator, such as a pressure reducer (not shown), arranged upstream and/or downstream of the valve means 7, in order to determine the flow rate of NO-based gas circulating in the NO supply apparatus 1. The flow meter can be of the pressure-differential type, the hot-wire type or some other type. It cooperates with the control means in order to provide them, once again, with measurements of the NO/N₂ flow rate, these measurements being processed by the control means 8 in order to ensure an efficient delivery of NO as a function in particular of the flow rate of O₂-based gas supplied by the medical ventilator 50.

Usually, the NO supply apparatus 1 also comprises a graphical user interface (GUI) comprising a graphical display screen 4, preferably a touch screen, i.e. a touch panel, serving to display various information items or data, icons, curves, alerts, etc., and also virtual selection keys and/or panes or windows, in particular for making choices, selections or for entering information, such as desired values (e.g. flow rate, dosage of NO, etc.), or any other information or data useful to the healthcare personnel. The display is preferably in colour, but it can also be in black and white.

The control means 8 of the NO supply apparatus 1 comprise, for example, an electronic control card and a microprocessor-based control unit 9, typically a microcontroller or the like. The control means 8 make it possible to adjust or control all the electromechanical elements of the apparatus 1. More precisely, the control card preferably integrates the control unit and is configured to control and also to analyse the signals coming from the various components, such as the sensors, etc.

The electrical power for the NO supply apparatus 1, in particular for the components requiring electrical current in order to operate, such as the control means, the graphical display screen 4, etc., is provided conventionally by an electrical current source and/or electrical supply means (not shown), for example a connection to the mains current (110/220V), such as an electrical cord and connection socket, and/or one or more electric, preferably rechargeable, batteries, and/or a current transformer. The electrical power supply to the medical ventilator 50 is ensured in a similar manner, in particular by a connection to the mains current or by an internal battery.

Finally, the installation 100 also comprises a gas sampling line 60 which fluidically connects the inspiratory branch 21 to the NO supply apparatus 1. It is fluidically connected (at 61) to the gas feed line 21, between the humidifier 30 and the junction piece 25, i.e. the Y-piece, typically in the immediate vicinity of the junction piece 25, and also to an inlet port 62 of the NO supply device 1, for example a port 62 carried by a connector, coupling or the like, for connecting the gas sampling line 60, such as a flexible hose or the like, to a gas analysis line 111 equipped with sensors 112 of an internal gas analyser 110, as detailed below.

The gas sampling line 60 makes it possible to take gas samples from the inspiratory branch 21 of the patient circuit 20 and to convey them to the NO supply device 1 where they are analysed in an internal gas analyser 110, that is to say within a gas analysis line 111 comprising measurement means, such as one or more sensors 112, typically one or more electrochemical cells for example, electrically connected to the control means 8, in order to verify the conformity of the analysed gas samples.

In particular, it should be verified that the composition of the final gas conforms with that of the desired NO/N₂/O₂ gas mixture to be administered to the patient, in particular in order to ensure that it does not contain excessive amounts of toxic NO₂ species, that its oxygen content is not hypoxic, that it does not have an excessively high NO₂ content, and that its NO content corresponds to the desired dosage, i.e. the dose of NO to be administered by inhalation that is usually chosen by the healthcare personnel, i.e. a doctor or the like.

In other words, the gas sampling line 60 makes it possible to monitor the composition of the final gas mixture, i.e. of the NO/N₂/O₂ gas mixture, in order to ensure that the contents of NO and O₂ species are in accordance with those desired, and that the content of toxic NO₂ species does not exceed a few ppmv.

This conformity check is conventionally carried out by dedicated measuring means, typically NO₂, NO and O₂ sensors 112, for example electrochemical cells or the like, which themselves have to be calibrated periodically, for example every week. This calibration is carried out by initiating a predefined calibration procedure stored in the storage means of the apparatus 1, such as a flash memory or the like.

The control means 8 of the apparatus 1 are also configured to recover and process, i.e. analyse, the signals coming from the various sensors 112 of the gas analyser 110, which is arranged in the apparatus 1, and to act in response to these signals, in particular to calibrate the sensors.

According to the invention, in order to minimize the risk of the healthcare personnel, i.e. the care team or teams, such as doctors, nurses or others, failing to initiate all or some of the checks that are essential for the proper functioning of the NO supply apparatus 1, in particular the regular calibration of the sensors 112, that is to say of the monitoring cells, typically NO and NO₂ sensors 112, of the gas analysis line 111.

To do this, the control means 8, which are electrically connected to the sensor or sensors 112 of the gas analysis line 111, i.e. one or more NO and NO₂ sensors, are configured to control, at a given time frequency, for example once a day, i.e. every 24 hours, a display, on the graphical display screen 4, of at least one item of information relating to one or more safety checks to be carried out, typically a reminder of a calibration to be carried out, and of a first virtual selection key 81, i.e. a first tactile key, the actuation of which by the user, that is to say the nursing personnel, causes the control means 8 to initiate a safety check procedure, i.e. a stored procedure, chosen from among those displayed by the graphical display screen 4, in particular a calibration procedure to be preferably performed on a daily basis, that is to say at least once a day.

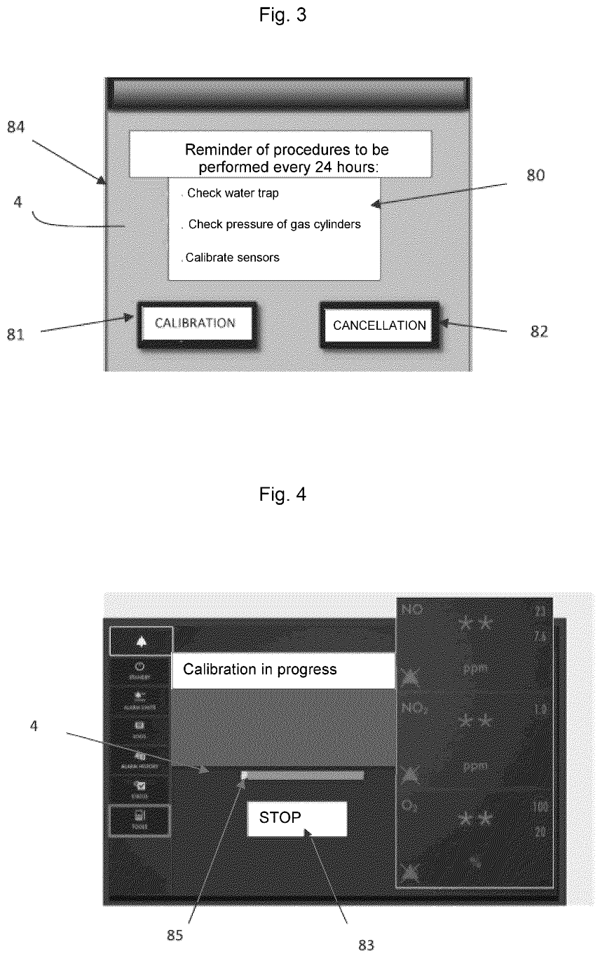

FIG. 3 shows an example of the display of information 80 relating to the safety checks to be carried out, namely here checking the water trap of the apparatus 1 and the pressure of the gas in the cylinders, in particular the pressure of the NO/N₂ gas mixture, and calibrating the sensors of the gas analysis line, and of a first virtual selection key 81 (CALIBRATION key), on the graphical display screen 4 of the NO supply apparatus 1, serving to initiate a calibration procedure, and preferably also a second virtual selection key 82 (CANCEL key) serving here to delay or cancel the initiation, i.e. start, of the calibration procedure.

Preferably, these displays are shown in a pop-up window 84 appearing on the graphical display screen 4.

The item or items of information 80 relating to safety checks to be carried out, in particular the calibration of the sensors, and the first and second virtual selection keys 81, 82 are displayed automatically on the graphical display screen at a given time frequency, typically at least once a day, i.e. at least every 24 hours. This is done by the control means 8, which integrate or cooperate with a time counter, for example a time counter integrated in the microprocessor 9.

The information 80 relating to safety checks to be carried out is preferably stored in the storage means of the apparatus 1, such as a flash memory or any other type of computer memory.

When the users see the item or items of information 80 relating to the safety checks to be carried out, in particular the calibration of the sensors, and the first and optionally second virtual selection keys 81, 82 appearing on the graphical display screen 4, in particular within a pop-up window, they can decide:
either to delay or prevent/reject the initiation of the calibration procedure by pressing the second virtual selection key 82, which is configured to prevent, i.e. not initiate, the calibration procedure,
or to initiate the calibration procedure by pressing the first virtual selection key 81.

Hence, if the first virtual selection key 81 is pressed, then a first signal (i.e. calibration initiation signal) is transmitted to the control means 8 which, in response to this press/selection by the user, then command a discontinuation of the display of the item or items of information 80 relating to the safety checks to be carried out, e.g. the calibration of the sensors, and of the first and second virtual selection keys 81, 82, and they also initiate a procedure of calibration of the sensors of the gas analysis line of the apparatus 1.

In this case, the item or items of information 80 relating to the safety checks to be carried out, e.g. the calibration of the sensors, and the first and optionally second keys will only be redisplayed at the end of the given time frequency, for example 1 to 4 times a day, especially within a new pop-up window.

However, according to an embodiment illustrated in FIG. 4, once a procedure of calibration of the sensors has been initiated, after pressing the first virtual selection key 81, the apparatus 1 can be configured to display, on the graphical display screen 4, a third virtual selection key 83 (STOP key), also actuatable by the user pressing it, and the actuation of which key generates a calibration procedure cancellation signal, which is transmitted to the control means 8 which, in response to this cancellation signal, then command that the calibration procedure that has started be stopped. This therefore enables the user to stop a calibration procedure, for example if they have made a mistake by unintentionally initiating such a procedure or, moreover, if the procedure has to be interrupted because there is an urgent need for the apparatus 1.

In other words, the control means can also be configured to control a display, on the graphical display screen 4, of a third virtual selection key 83, the actuation of which by the user causes a discontinuation of a calibration procedure that has started, that is to say that has started after the user has pressed the first virtual selection key.

Advantageously, a time graph 85 such as a bar graph, hourglass or similar is also displayed, showing the time that has elapsed since the start of the calibration procedure and/or the time remaining before the termination, i.e. end, of the calibration procedure. This helps the user determine whether or not to interrupt the calibration procedure by pressing the third virtual selection key 83.

Conversely, if the user presses the second virtual selection key 82, then a second signal (i.e. signal for non-initiation of calibration or delayed calibration) is transmitted to the control means 8 which, in response to this press/selection by the user, then command a discontinuation of the display of the information 80 relating to the safety checks to be carried out, e.g. calibration of the sensors, and of the first and second virtual selection keys 81, 82, but without initiating calibration.

In this case, the item or items of information 80 relating to the safety checks to be carried out, e.g. the calibration of the sensors, and the first and preferably second virtual selection keys 81, 82 then disappear from the display screen 4 and will be redisplayed there only after a given period of time, for example after 1 or 2 hours, or another period of time, or only at the given time frequency, that is to say after 24 hours for example.

The display on the display screen 4 can be in colour or in black and white, depending on what is selected by the user from the menus of the apparatus 1.

The NO supply apparatus 1 of the invention can be used in a gas administration installation 100, for example that of FIG. 1, in order to administer nitric oxide (NOi), i.e. the final NO/O₂/N₂ mixture obtained, via a mask or tracheal intubation tube to a person or persons, i.e. patients, who require it to treat their pulmonary pathology, typically one or more patients suffering from acute pulmonary arterial hypertension, in particular to dilate their pulmonary vessels and/or increase their oxygenation by improving pulmonary gas exchange, in particular to treat pulmonary arterial hypertension of the newborn (PPHN), acute respiratory distress syndrome (ARDS) observed mainly in adults, or pulmonary hypertension (PH) in cardiac surgery, observed in adults or children.

1. Apparatus (1) for supplying an NO-containing gas, comprising:
an internal gas circuit (6) for conveying a flow of NO-containing gas, comprising valve means (7),
control means (8) with a microprocessor (9),
a tactile graphical display screen (4) controlled by the control means (8), and
a gas analysis line (111) comprising at least one sensor (112) electrically connected to the control means (8),
characterized in that the control means (8) are configured to control, at a given time frequency, a temporary display, on the graphical display screen (4), of at least a first virtual selection key (81), the actuation of which by a user causes initiation, by the control means (8), of a procedure of calibration of the one or more sensors (112) of the gas analysis line (111).

2. Apparatus according to claim 1, characterized in that the first virtual selection key (81) cooperates with the control means (8), in order to supply them with at least one control signal corresponding to a choice made by the user corresponding to the initiation of the sensor calibration procedure.

3. Apparatus according to claim 1, characterized in that the control means (8) are further configured to control a display, on the graphical display screen (4), of at least one item of information (80) relating to one or more safety checks to be carried out, in particular of the sensor calibration procedure.

4. Apparatus according to claim 3, characterized in that the control means (8) are configured to control the display of said at least one item of information (80) and/or of the first virtual selection key (81) at a time frequency of at least once a day.

5. Apparatus according to claim 1, characterized in that the control means (8) are configured to further control a display of a second virtual selection key (82) simultaneously or successively to said first virtual selection key (81), said second virtual selection key (82) being configured to cancel or reject said sensor calibration procedure in response to a user actuation of the second virtual selection key (82).

6. Apparatus according to claim 1, characterized in that the control means (8) are configured to control a simultaneous display of said at least one item of information (80) and of the first and optionally second virtual selection keys (81, 82).

7. Apparatus according to claim 1, characterized in that the control means (8) control the valve means (7) in order to control the flow of gas in the internal gas circuit (6), so as to permit or stop any circulation of gas in said internal gas circuit (6).

8. Apparatus according to claim 1, characterized in that said at least one sensor (112) comprises one or more electrochemical cells.

9. Apparatus according to claim 6, characterized in that the control means (8) are configured to control the simultaneous display of said at least one item of information (80) and of the first and optionally second virtual selection keys (81, 82) within a pop-up window (84).

10. Apparatus according to claim 4, characterized in that the control means (8) are configured to control the display of said at least one item of information (80) and/or of the first virtual selection key (81) at a time frequency of 1 to 4 times a day, preferably 1 or 2 times a day.

11. Apparatus according to claim 3, characterized in that it comprises storage means configured to store the safety check procedure or procedures, in particular the sensor calibration procedure.

12. Apparatus according to claim 1, characterized in that the control means (8) are further configured to control a display, on the graphical display screen (4), of a third virtual selection key (83), the actuation of which by the user causes a discontinuation of a calibration procedure that has started.

13. Apparatus according to claim 1, characterized in that the control means (8) are further configured to control a display, on the graphical display screen (4), of a time graph (85) representing the time that has elapsed since the start of the calibration procedure and/or the time remaining before the termination of the calibration procedure, in particular a time graph representing a bar graph or an hourglass.

14. Apparatus according to claim 12, characterized in that the control means (8) are further configured to control a simultaneous display, on the graphical display screen (4), of the third virtual selection key (83) and of the time graph (85).

15. Installation (100) for administering gas to a patient, comprising:
at least one gas source (10) containing gaseous NO, in particular an NO/N₂ gas mixture,
a gas supply apparatus (1) according to any one of the preceding claims, fed with NO-containing gas by said at least one gas source (10),
a medical ventilator (50) for supplying an oxygen-containing gas, and
a gas feed line (21) supplied with gas by the gas supply apparatus (1) and by the medical ventilator (50).
